(19) United States
(12) Patent Application Publication
(10) Pub. No.: US 2023/0343455 A1
(43) Pub. Date: Oct. 26, 2023

(54) MEDICAL IMAGE DIAGNOSIS ASSISTANT APPARATUS AND METHOD FOR GENERATING AND VISUALIZING ASSISTANT INFORMATION BASED ON DISTRIBUTIONS OF SIGNAL INTENSITIES IN MEDICAL IMAGES

(71) Applicant: CORELINE SOFT CO,, LTD., Seoul (KR)

(72) Inventors: Sunggoo KWON, Guri-si (KR); Jin Seol KIM, Seoul (KR); Hyunwoo KIM, Seoul (KR); Seyeon JO, Bucheon-si (KR); Chan Mi PARK, Seoul (KR)

(21) Appl. No.: 18/307,444

(22) Filed: Apr. 26, 2023

(30) Foreign Application Priority Data
Apr. 26, 2022 (KR) 10-2022-0051650
Sep. 19, 2022 (KR) 10-2022-0118135

(51) Int. Cl. G16H 50/20; G06T 7/00; G06T 11/20; G06T 11/60; G16H 30/40

(52) U.S. Cl. CPC G16H 50/20; G06T 7/0012; G06T 11/206; G06T 11/60; G16H 30/40; G06T 2207/30096

(57) ABSTRACT

A medical image diagnosis assistant apparatus is configured to acquire information about at least one target region in a medical image; acquire distribution information about a distribution of signal intensity values within the target region; generate first assistant information based on a first threshold for the signal intensity values within the target region; and generate second assistant information based on the distribution information and the first assistant information.

### CROSS-REFERENCE TO RELATED APPLICATION

This application claims priority from Korean Patent Application Nos. 10-2022-0051650 and 10-2022-0118135 filed on Apr. 26, 2022 and Sep. 19, 2022, which are incorporated herein by reference in their entireties.

### TECHNICAL FIELD

The present invention relates to an apparatus and method for assisting diagnosis using medical images. More particularly, the present invention relates to technology for analyzing medical images and visualizing the results of the analysis in order to assist diagnosis using the medical images.

### BACKGROUND ART

The contents described in this section merely provide information about the background art of the present invention and do not constitute prior art.

Currently, medical images such as computed tomography (CT) images are widely used to make diagnoses by analyzing lesions. For example, CT images of the chest are frequently used for diagnosis because abnormalities in the inside of the body, such as the lungs, the bronchi, and the heart, can be observed.

Some findings that can be diagnosed through chest CT images are not so easy to read that even radiologists can distinguish their features and shapes only after years of training, with the result that human doctors may easily overlook them. In particular, when the level of difficulty in reading is high, as in the case of a lung nodule, a doctor may not see it even if he or she pays a high degree of attention, which may cause a problem.

In order to assist in the reading of images that humans may easily overlook, the need for computer-aided diagnosis (CAD) has emerged. However, conventional CAD technology only assists doctors in making decisions in a considerably limited area.

The reading of a lesion using CAD may include a process of first specifying a region suspected of being a lesion and evaluating a score (e.g., confidence, malignity, and/or the like) for the region. For example, when a plurality of nodules are found in a lung region, it will be necessary to specify a nodule that is expected to be highly malignant among them and determine a future treatment plan.

However, since there are the plurality of nodules, it is difficult to be aware of a nodule having the highest degree of malignancy among them before reading. Furthermore, in many cases, diagnosis starts to be performed from nodules that are not expected to be malignant or that are not actually malignant, resulting in a decrease in reading efficiency. In addition, since it is difficult to know which nodule is an actual nodule before reading and reliability is not high, reading efficiency is low even if diagnosis starts to be performed from a part that is not expected to be an actual nodule.

Korean Patent No. 10-1943011 entitled “Method for Assisting Reading of Medical Images of Subject and Apparatus Using the Same,” which is a prior art document, discloses a method and an apparatus using the method that can improve reading efficiency by introducing a score evaluation method into a conventional lesion detection system and allowing lesions with the highest score (e.g., reliability, malignancy, and/or the like) to be read first out of detected lesions.

In Korean Patent No. 10-1943011, there is disclosed a configuration in which, when a plurality of lesions are detected for a single type of disease, a list arranged from lesions having the highest score in terms of reliability, malignancy, and/or the like is displayed within a single display setting and an image associated with a corresponding lesion is displayed when a user selects the lesion from the list.

However, even when information about a lesion detected through an artificial neural network is visualized together with a score such as reliability, malignancy and/or the like given by the artificial neural network, it is difficult to know the grounds for the calculation of reliability, malignancy and/or the like from the outside because the inside of the artificial neural network is similar to a black box. Therefore, a medical professional, who is a user, cannot be provided with a means of verifying the inference results of the artificial neural network.

### SUMMARY

The present invention has been conceived to overcome the above-described problems, and an object of the present invention is to provide a means, by which a medical professional can verify the results of detection of a specific organ, lesion, or finding, as assistant information.

An object of the present invention is to provide a means, by which a medical professional can obtain additional information about the presence/absence, progress, and severity of a disease for a specific organ, lesion, or finding, as assistant information.

An object of the present invention is to, in a state in which a specific organ is segmented or a lesion or finding is detected, and is then visualized, provide information about whether a disease is actually present in the segmented organ or the detected lesion or finding, whether the disease is in progress, or whether the disease is severe together with assistant information that a medical professional, which is a user, can be aware of.

An object of the present invention is to provide a function that facilitates the follow-up examination of the transition of a specific lesion or organ over time.

An object of the present invention is to provide visualization information that is effective for representing the type or state of a region of interest (ROI).

An object of the present invention is to provide visualization information that is effective for representing a change in the type or state of an ROI.

According to an embodiment of the present invention, there is provided a medical image diagnosis assistant apparatus comprising a processor, wherein the processor is configured to: acquire information about at least one target region in a medical image; acquire distribution information about a distribution of signal intensity values within the target region; generate first assistant information based on a first threshold for the signal intensity values within the target region; and generate second assistant information based on the distribution information and the first assistant information.

The signal intensity values within the target region may be classified into a first interval corresponding to a first state and a second interval corresponding to a second state based on the first threshold.

The second assistant information may comprise at least one of first interval distribution information corresponding to the first interval of the signal intensity values within the target region and second interval distribution information corresponding to the second interval of the signal intensity values within the target region.

The second assistant information further may comprise at least one of a first visualization element representative of the first interval distribution information and a second visualization element representative of the second interval distribution information.

The second assistant information may comprise at least one of first interval distribution quantification information corresponding to the first interval of the signal intensity values within the target region and second interval distribution quantification information corresponding to the second interval of the signal intensity values within the target region.

The first interval distribution quantification information may comprise at least one of a percentile, maximum value, minimum value, mean value, mode value, and median value of pixels/voxels of signal intensity values within the target region corresponding to the first interval.

The second interval distribution quantification information may comprise at least one of a percentile, maximum value, minimum value, mean value, mode value, and median value of pixels/voxels of signal intensity values within the target region corresponding to the second interval.

The processor may be further configured to generate at least one of: first overlay visualization information on the medical image for at least one first sub-region within the target region corresponding to the first interval; and second overlay visualization information on the medical image for at least one second sub-region within the target region corresponding to the second interval.

The processor may be further configured to: generate the first overlay visualization information when a user input for the first interval associated with the second assistant information is recognized; and generate the second overlay visualization information when a user input for the second interval associated with the second assistant information is recognized.

The first threshold may be associated with at least one of a presence/absence, progress, and severity of a disease associated with the target region.

The information about the target region may be segmentation information about a boundary of the target region.

The target region may be a finding region detected in association with a disease or lesion in the medical image.

The target region may be a region obtained as a result of segmentation of an anatomical structure in the medical image.

The processor may be further configured to generate the first assistant information further including a second threshold value for the signal intensity values within the target region.

The signal intensity values within the target region may be classified into a first interval corresponding to a first state, a second interval corresponding to a second state, and a third interval corresponding to a third state based on the first threshold and the second threshold.

The distribution information may be histogram information corresponding to a distribution of signal intensity values of pixels/voxels within the target region.

According to an embodiment of the present invention, there is provided a medical image diagnosis assistant apparatus comprising a processor, wherein the processor is configured to: acquire information about at least one first target region in a first medical image acquired at a first time for a subject; acquire information about at least one second target region in a second medical image acquired at a second time for the subject; acquire first distribution information about a distribution of signal intensity values within the first target region; acquire second distribution information about a distribution of signal intensity values within the second target region; and generate visualization information based on the first distribution information and the second distribution information.

The processor may be further configured to generate first assistant information based on a first threshold for signal intensity values in the first area and the second area.

The signal intensity values within the first region and the second region may be classified into a first interval corresponding to a first state and a second interval corresponding to a second state based on the first threshold.

The processor may be further configured to acquire at least one of first interval distribution information corresponding to the first interval for the signal intensity values within the first region and second interval distribution information corresponding to the second interval for the signal intensity values within the first region based on the first distribution information.

The processor may be further configured to generate, as a part of the visualization information, second assistant information including the first assistant information while including at least one of the first interval distribution information and the second interval distribution information.

The processor may be further configured to acquire at least one of third interval distribution information corresponding to the first interval for the signal intensity values within the second region and fourth interval distribution information corresponding to the second interval for the signal intensity values within the second region based on the second distribution information.

The processor may be further configured to generate, as a part of the visualization information, third assistant information including the first assistant information while including at least one of the third interval distribution information and the fourth interval distribution information.

The processor may be further configured to generate first assistant information based on a first threshold for signal intensity values in the first area and the second area.

The signal intensity values within the first region and the second region may be classified into a first interval corresponding to a first state and a second interval corresponding to a second state based on the first threshold.

The processor may be further configured to acquire at least one of first interval distribution quantification information corresponding to the first interval for the signal intensity values within the first region and second interval distribution quantification information corresponding to the second interval for the signal intensity values within the first region based on the first distribution information.

The processor may be further configured to generate, as a part of the visualization information, second assistant information including the first assistant information while including at least one of the first interval distribution quantification information and the second interval distribution quantification information.

The processor may be further configured to acquire at least one of third interval distribution quantification information corresponding to the first interval for the signal intensity values within the second region and fourth interval distribution quantification information corresponding to the second interval for the signal intensity values within the second region based on the second distribution information.

The processor may be further configured to generate, as a part of the visualization information, third assistant information including the first assistant information while including at least one of the third interval distribution quantification information and the fourth interval distribution quantification information.

According to an embodiment of the present invention, there is provided a medical image diagnosis assistant method being performed by a computing system including a processor, the medical image diagnosis assistant method comprising: acquiring information about at least one target region in a medical image; acquiring distribution information about a distribution of signal intensity values within the target region; generating first assistant information based on a first threshold for the signal intensity values within the target region; and generating second assistant information based on the distribution information and the first assistant information.

The signal intensity values within the target region may be classified into a first interval corresponding to a first state and a second interval corresponding to a second state based on the first threshold.

The generating second assistant may comprise generating the second assistant information including at least one of first interval distribution information corresponding to the first interval of the signal intensity values within the target region and second interval distribution information corresponding to the second interval of the signal intensity values within the target region.

The signal intensity values within the target region may be classified into a first interval corresponding to a first state and a second interval corresponding to a second state based on the first threshold.

The generating second assistant may comprise generating the second assistant information including at least one of first interval distribution quantification information corresponding to the first interval of the signal intensity values within the target region and second interval distribution quantification information corresponding to the second interval of the signal intensity values within the target region.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing a medical image follow-up examination and a target region as a subject of the follow-up examination according to an embodiment of the present invention;

FIG. 2 is a diagram showing an example of assistant information visualized by a medical image diagnosis assistant apparatus according to an embodiment of the present invention;

FIG. 3 is a diagram showing another example of assistant information visualized by a medical image diagnosis assistant apparatus according to an embodiment of the present invention;

FIG. 4 is a diagram showing an example of assistant information visualized for medical image follow-up examination by a medical image diagnosis assistant apparatus according to an embodiment of the present invention;

FIG. 5 is a diagram showing an example of assistant information visualized for medical image follow-up examination by a medical image diagnosis assistant apparatus according to an embodiment of the present invention;

FIG. 6 is a diagram showing an example of a medical image visualized in association with assistant information for medical image follow-up examination by a medical image diagnosis assistant apparatus according to an embodiment of the present invention;

FIG. 7 is a diagram showing an example of a medical image visualized in association with assistant information for medical image follow-up examination by a medical image diagnosis assistant apparatus according to an embodiment of the present invention;

FIG. 8 is an operational flowchart showing a medical image diagnosis assistant method according to an embodiment of the present invention;

FIGS. 9 and 10 are operational flowcharts each showing in detail an example of a partial process of the medical image diagnosis assistant method of FIG. 8;

FIG. 11 is an operational flowchart showing a medical image diagnosis assistant method according to an embodiment of the present invention;

FIGS. 12 to 15 are operational flowcharts each showing in detail an example of a partial process of the medical image diagnosis assistant method of FIG. 11;

FIG. 16 is a drawing showing an example of medical image follow-up examination and assistant information according to an embodiment of the present invention;

FIG. 17 is a block diagram showing a generalized medical image diagnosis assistant apparatus or computing system capable of performing at least a part of the processes of FIGS. 1 to 16 according to an embodiment of the present invention; and

FIG. 18 is a conceptual diagram including a processor and an artificial neural network as the internal structure of a generalized medical image diagnosis assistant apparatus or computing system according to an embodiment of the present invention.

### DETAILED DESCRIPTION OF THE DISCLOSURE

Other objects and features of the present invention in addition to the above-described objects will be apparent from the following description of embodiments to be given with reference to the accompanying drawings.

The embodiments of the present invention will be described in detail below with reference to the accompanying drawings. In the following description, when it is determined that a detailed description of a known component or function may unnecessarily make the gist of the present invention obscure, it will be omitted.

Relational terms such as first, second, and the like may be used for describing various elements, but the elements should not be limited by the terms. These terms are only used to distinguish one element from another. For example, a first component may be named a second component without departing from the scope of the present disclosure, and the second component may also be similarly named the first component. The term “and/or” means any one or a combination of a plurality of related and described items.

When it is mentioned that a certain component is “coupled with” or “connected with” another component, it should be understood that the certain component is directly “coupled with” or “connected with” to the other component or a further component may be disposed therebetween. In contrast, when it is mentioned that a certain component is “directly coupled with” or “directly connected with” another component, it will be understood that a further component is not disposed therebetween.

The terms used in the present disclosure are only used to describe specific exemplary embodiments, and are not intended to limit the present disclosure. The singular expression includes the plural expression unless the context clearly dictates otherwise. In the present disclosure, terms such as ‘comprise’ or ‘have’ are intended to designate that a feature, number, step, operation, component, part, or combination thereof described in the specification exists, but it should be understood that the terms do not preclude existence or addition of one or more features, numbers, steps, operations, components, parts, or combinations thereof.

Unless otherwise defined, all terms (including technical and scientific terms) used herein have the same meaning as commonly understood by one of ordinary skill in the art to which this disclosure belongs. Terms that are generally used and have been in dictionaries should be construed as having meanings matched with contextual meanings in the art. In this description, unless defined clearly, terms are not necessarily construed as having formal meanings.

As to recent medical images such as CT or MRI images, a series of medical images is acquired through a single acquisition process, and the series of medical images is not limited to a single type of lesion but may also be used to detect various types of lesions.

Deep learning/CNN-based artificial neural network technology, which has recently developed rapidly, is considered for the purpose of identifying a visual element that is difficult to identify with the human eye when it is applied to the imaging field. The fields of application of the above technology are expected to expand to various fields such as security, medical imaging, and non-destructive testing.

For example, in the medical imaging field, there are cases where a tissue in question is not immediately diagnosed as a cancer tissue in a biopsy state but whether it is a cancer tissue is determined only after being monitored from a pathological point of view. Although it is difficult to confirm whether a corresponding cell is a cancer tissue in a medical image with the human eye, there is an expectation that the application of artificial neural network technology may acquire more accurate prediction results than observation with the human eye.

It is expected that this artificial neural network technology is applied and performs the analysis process of detecting a disease or lesion difficult to identify with the human eye in a medical image, segmenting a region of interest such as a specific tissue, and measuring the segmented region.

Meanwhile, even if a technology is known prior to the filing date of the present disclosure, it may be included as part of the configuration of the present disclosure when necessary, and will be described herein without obscuring the spirit of the present disclosure. However, in describing the configuration of the present disclosure, a detailed description on matters that can be clearly understood by those skilled in the art as a known technology prior to the filing date of the present disclosure may obscure the purpose of the present disclosure, so excessively detailed description on the known technology will be omitted.

Descriptions of some known items may be replaced by providing notification that the items are known to those of ordinary skill in the art via the related art literature, including Korean Patent No. 10-2270934 entitled “Medical Image Reading Assistant Apparatus and Method for Providing Representative Images Based on Medical Artificial Neural Network,” Korean Patent No. 10-2283673 entitled “Medical Image Reading Assistant Apparatus and Method for Adjusting Threshold of Diagnostic Assistant Information Based on Lesion Follow-up Examination,” Korean Patent No. 10-1943011 entitled “Method for Assisting Reading of Medical Images of Subject and Apparatus Using the Same,” Korean Patent No. 10-1887194 entitled “Method for Assisting Reading of Medical Images of Subject and Apparatus Using the Same,” Korean Patent No. 10-1818074 entitled “Artificial Intelligence-based Medical Automatic Diagnosis Assistant Method and System Thereof,” etc., that are cited therein.

Some of the contents disclosed in these related art documents are related to the objects to be achieved by the present invention, and some of the solutions adopted by the present invention are applied to these related art documents in the same manner.

In the above-described related documents, lesion candidates are detected using an artificial neural network and classified, and findings are then generated. Each of the findings may include diagnosis assistance information, and the diagnosis assistance information may include quantitative measurements such as the probability that the finding corresponds to an actual lesion, the confidence of the finding, and the malignity, size and volume of the corresponding one of the lesion candidates to which the findings correspond.

In medical image reading assistance using an artificial neural network, each finding may include numerically quantified probability or confidence as diagnosis assistance information. Since all findings may not be provided to a user, the findings are filtered by applying a predetermined threshold, and only passed findings are provided to the user.

Diagnosis using a medical image refers to a process in which a medical professional identifies a disease or lesion that has occurred in a patient. In this case, prior to diagnosis using a medical image, a medical professional analyzes the medical image and detects a disease or lesion appearing in the medical image. A primary opinion on the detection of a disease or lesion on a medical image is referred to as “findings,” and the process of deriving findings by analyzing a medical image is referred to as “reading.”

Diagnosis using a medical image is made in such a manner that a medical professional analyzes the findings, derived through the process of reading the medical image, again. In this process, role division is frequently performed in such a manner that a radiologist reads a medical image and derives findings and a clinician derives a diagnosis based on a reading result and the findings.

In this workflow, the ANN and/or automation software may assist at least a part of reading of radiologists, generating of findings, and/or diagnosis of clinicians.

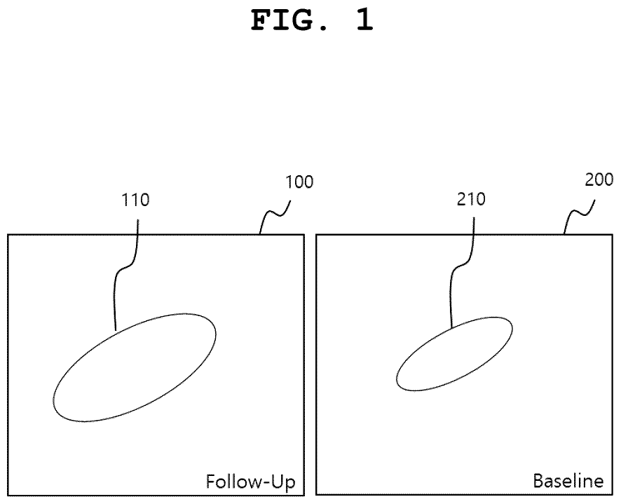

In the following description to be given in conjunction with FIGS. 1 to 18, the descriptions of items that are considered to be well-known techniques widely known in the technical field of the present invention may be omitted as necessary in order to prevent the gist from being obscured, or may be replaced by citing the related art documents.

Furthermore, some or all of the configurations disclosed in the related art documents cited above and to be cited later may be related to some of the objects to be achieved by the present invention, and some of the solutions adopted by the present invention may be borrowed from the related art documents.

Only the items included to embody the present invention among the items disclosed in the related art documents will be considered to be parts of the components of the present invention.

Details of the present invention will be described below in conjunction with the embodiments of FIGS. 1 to 18.

FIG. 1 is a diagram showing a medical image follow-up examination and a target region as a subject of the follow-up examination according to an embodiment of the present invention.

Referring to FIG. 1, there are shown a first medical image 100, which is a follow-up examination image acquired at a current time, and a second medical image 200, which is a baseline image acquired at a past time.

A first target region 110 may be identified and marked in the first medical image 100, and a second target region 210 may be identified and marked in the second medical image 200.

It is assumed that it has already been recognized that the first target region 110 and the second target region 210 correspond to each other. The correspondence between the first target region 110 and the second target region 210 may be acquired in advance by registration between the first medical image 100 and the second medical image 200.

A medical image diagnosis assistant apparatus according to an embodiment of the present invention may provide a function of performing follow-up examination on the second target region 210, which is an ROI identified in the second medical image 200, i.e., a previous image, and the first target region 110, which is an ROI identified in the first medical image 100, i.e., a follow-up image. The apparatus of the present invention may combine and provide a follow-up examination function and a histogram visualization function based on the distribution of signal intensity values of the ROI of a previous image and the distribution of signal intensity values of the ROI of a subsequent image.

When the ROI is an organ or a lesion, the medical image diagnosis assistant apparatus may provide the histogram visualization function as part of a menu capable of checking whether a process of segmenting the organ or lesion is appropriate.

When the ROI is not an organ but a lesion, the medical image diagnosis assistant apparatus may provide a histogram based on the distribution of signal intensity values of pixels/voxels within the lesion.

The medical image diagnosis assistant apparatus may provide an appropriate window for signal intensity values (brightness values) for a CT histogram effective in assisting diagnosis for a specific organ or lesion.

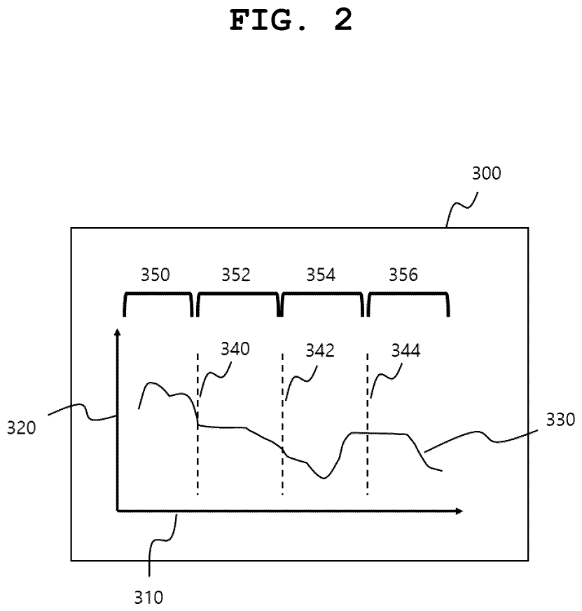

FIG. 2 is a diagram showing an example of assistant information visualized by a medical image diagnosis assistant apparatus according to an embodiment of the present invention.

Although not shown in FIG. 2, a medical image diagnosis assistant apparatus according to an embodiment of the present invention may be a computing system including a processor. The operation of the computing system of the present invention may be performed under the control or management of the processor.

FIG. 2 may be applied to either of the first medical image 100 and second medical image 200 of FIG. 1. For convenience of description, it is assumed that FIG. 2 corresponds to the first medical image 100.

According to an embodiment of the present invention, the apparatus: acquires information about at least one first target region 110 in the first medical image 100; acquires distribution information 330 about the distribution of signal intensity values within the first target region 110; generates first assistant information 340 based on a first threshold for the signal intensity values within the first target region 110; and generates second assistant information 300 based on the distribution information 330 and the first assistant information 340.

The distribution information 330 may be visualized along with a horizontal axis 310 corresponding to signal intensity values and a vertical axis 320 corresponding to the numbers and/or distributions of pixels/voxels having signal intensity values. The signal intensity values may be the brightness values of a CT scan image or an X-ray image, and may be expressed in Hounsfield units (HU). The distribution information 330 may be a histogram corresponding to the signal intensity values of pixels/voxels in the first target region 110.

According to an embodiment of the present invention, the first target region 110 may be a lesion. For example, the first target region 110 may be a lung nodule, a coronary artery calcification (CAC) region, or a lung tumor.

When the first target region 110 is a lung nodule, a user is assisted with a process of identifying the type of nodule by marking −100 HU and 100 HU reference lines as first assistant information 340 along with distribution information 330 for CT brightness values. The range of HU values on a horizontal axis plotted on a histogram is an embodiment specific to a corresponding lesion, and may be optimized within the interval of [−1000 HU, 500 HU] in the case of a lung nodule, for example.

In an embodiment in which the distribution of brightness values is visualized through a histogram, a user may be assisted in identifying the type or state of an ROI by providing one or more reference lines for brightness values. In this case, the results of the quantitative analysis of a plurality of brightness value intervals divided by one or more reference lines may be visualized together with the reference lines.

Alternatively, a medical image analysis module may determine the type or state of an ROI based on one or more reference lines, and the results of the determination may be visualized along with the reference lines. In this case, the results of the quantitative analysis of a plurality of brightness value intervals divided by one or more reference lines may be visualized together with the reference lines and the results of determination of the type or state of an ROI. In this case, the results of the determination may further include predictive/inferential information about a change in the type or state of an ROI.

For example, when the ROI is a lung nodule, a brightness value reference line that makes it easy to identify the type (solid, non-solid, or part-solid) of nodule may be provided.

The first assistant information 340 may be a reference line or an interval mark corresponding to a first threshold visualized together with the distribution information 330. The first threshold may be a threshold for signal intensity values.

The signal intensity values within the first target region 110 may be classified into a first interval 350 corresponding to a first state and a second interval 352 corresponding to a second state based on the first threshold.

The second assistant information 300 may include at least one of first interval distribution information corresponding to the first interval 350 of the signal intensity values within the first target region 110 and second interval distribution information corresponding to the second interval 352 of the signal intensity values within the first target region 110.

The first interval distribution information may be a segment or part belonging to the first interval 350 of the distribution information 330. The second interval distribution information may be a segment or part belonging to the second interval 352 of the distribution information 330.

The second assistant information 300 may include individual pieces of interval distribution information obtained by dividing the distribution information 330, which is a histogram, into individual intervals 350, 352, 354, and 356, and may also include first assistant information 340, 342, and 344 corresponding to thresholds for division into the individual intervals 350, 352, 354, and 356. The second assistant information 300 may be generated as visualization information in order to be provided to a user through a display or user interface. As the visualization information is visualized to the user, the second assistant information 300 may be provided to the user.

The second assistant information 300 may further include a first visualization element representing the first interval distribution information and a second visualization element representing the second interval distribution information. In other words, individual segments may be visualized using different visualization elements so that a histogram can be visually divided into individual intervals. The visualization elements may include colors, patterns, markers, line thicknesses, solid/dotted lines, and/or shapes.

The first threshold may be a value associated with at least one of the presence/absence, progress, and severity of a disease associated with the first target region 110. The first threshold may be a value applied to both the first target region 110 and the second target region 210.

The information about the first target region 110 may include segmentation information about the boundary of the first target region 110. In other words, the information about the first target region 110 may include boundary information obtained as a result of segmentation.

The target region may be the region of a finding detected in association with a disease or lesion in a medical image. For example, the lesion may include a nodule, a coronary artery calcification (CAC), a tumor, and the like. For example, the finding may include a low attenuation area (LAA) associated with a disease such as chronic obstructive pulmonary disease (COPD).

The target region may be a region obtained as a result of the segmentation of an anatomical structure in a medical image. An example of the anatomical structure is an organ. The organ may include a lung, the liver, the heart, a blood vessel, and the like.

Information about the target region of the present invention may be obtained by the inference of an artificial neural network.

Alternatively, information about the target region of the present invention may be obtained by the operation of a rule-based analysis engine.

An embodiment of the present invention may provide distribution information, first assistant information, and second assistant information to a user as means for verifying information about a target region, which is an inference result of an artificial neural network or an operation result of a rule-based analysis engine.

In this case, an embodiment of the present invention may generate second assistant information (including distribution information and first assistant information) as visualization information to be provided to a user.

In this case, an embodiment of the present invention may provide the visualization information to the user via a user interface such as a display.

An embodiment of the present invention may provide a user with second assistant information together with visualization information about a target region in a medical image.

In this case, an embodiment of the present invention may automatically provide a user with second assistant information together with visualization information about a target region, or may provide a user with second assistant information about a target region in response to a user input when the user input is recognized for visualization information about the target region.

In an embodiment of the present invention, the signal intensity values within the target region may be classified into a first interval corresponding to the first state of the pixels/voxels within the target region or a second interval corresponding to the second state thereof. In this case, the first state and the second state may be states distinguished from each other in association with at least one of the presence/absence, progress, and severity of a disease associated with the target region. The first state and the second state are states that each of the pixels/voxels within the target region may have, and may be states distinguished from each other in association with at least one of the presence/absence, progress, and severity of a disease.

Referring to FIG. 2, the apparatus may generate three pieces of first assistant information 340, 342, and 344 using a plurality of thresholds for the signal intensity values within the target region.

In this case, the signal intensity values within the target region may be classified into a first interval 350 corresponding to the first state, the second interval 352 corresponding to the second state, and the third interval 354 corresponding to the third state based on one or more thresholds. The individual intervals may be set to correspond to the states of the pixels/voxels within the target region. The states may be set differently for individual sub-regions within the target region in association with the presence/absence, progress, and/or severity of a disease associated with the target region. Interval information associated with the states may be provided as additional diagnostic information for the target region.

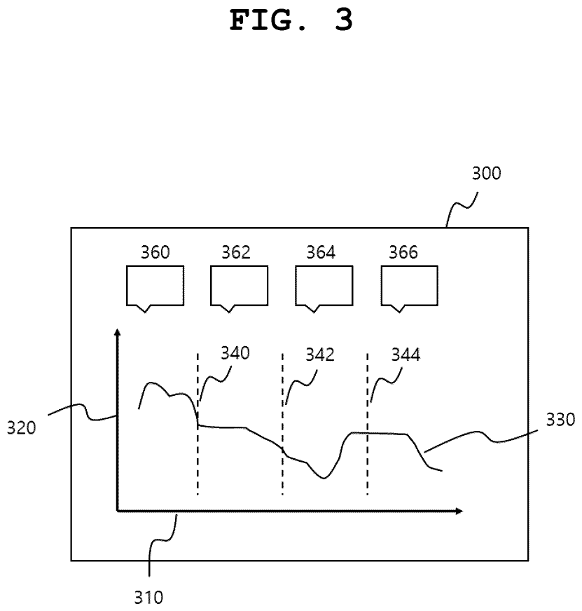

FIG. 3 is a diagram showing another example of assistant information visualized by a medical image diagnosis assistant apparatus according to an embodiment of the present invention.

Referring to FIG. 3, the second assistant information 300 may include at least one of first interval distribution quantification information 360, which is the quantification information of a distribution corresponding to the first interval of the signal intensity values of pixels/voxels within a target region, and second interval distribution quantification information 362, which is the quantification information of a distribution corresponding to the second interval of the signal intensity values of the pixels/voxels within the target region.

Pieces of interval distribution quantification information 360, 362, 364, and 366 may be pieces of statistical/quantification information for respective intervals of a histogram graph. The number and distribution of pixels/voxels corresponding to each of the intervals, or the ratio of the number of pixels/voxels corresponding to each interval to the number of pixels/voxels in the overall target region may be included as each of the pieces of distribution quantification information 360, 362, 364, and 366.

The first interval distribution quantification information 360 may include at least one of a percentage, a maximum value, a minimum value, a mean value, a mode value, and a median value associated with the number/distribution of pixels/voxels having signal intensity values corresponding to the first interval within the target region.

In the same manner, the second interval distribution quantification information 360 may include at least one of a percentage, a maximum value, a minimum value, a mean value, a mode value, and a median value associated with the number/distribution of pixels/voxels having signal intensity values corresponding to the second interval within the target region.

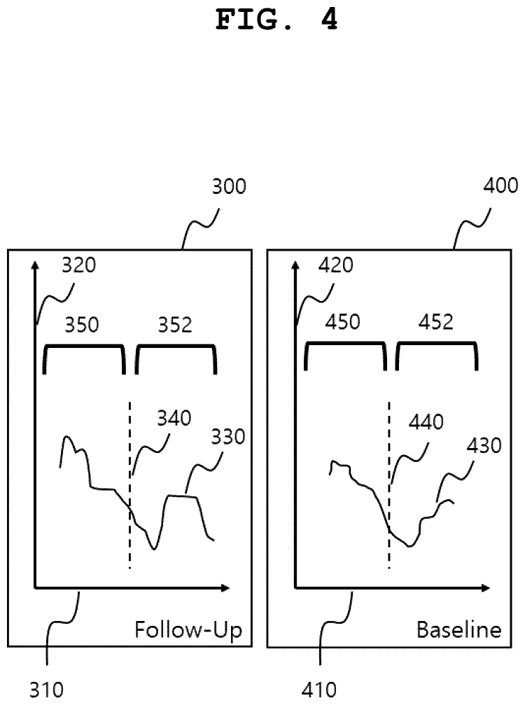

FIG. 4 is a diagram showing an example of assistant information visualized for medical image follow-up examination by a medical image diagnosis assistant apparatus according to an embodiment of the present invention.

A medical image diagnosis assistant apparatus according to an embodiment of the present invention may include a processor, and the operation of the apparatus may be performed under the control and management of the processor. The apparatus acquires information about the at least one first target region 110 in the first medical image 100 acquired at a first time (current time) for a subject, and also acquires information about the at least one second target region 210 in the second medical image 200 acquired at a second time (past time) for the subject. The second target region 210 corresponds to the first target region 110, and the correspondence between the two regions 110 and 210 may be acquired as a result of registration between the first medical image 100 and the second medical image 200.

The apparatus acquires first distribution information 330 about the distribution of signal intensity values within the first target region 110, and also acquires second distribution information 430 about the distribution of signal intensity values within the second target region 210.

The apparatus may generate visualization information including the first distribution information 330 and the second distribution information 430 so that the first distribution information 330 and the second distribution information 430 can be compared with each other. In this case, the visualization information does not necessarily refer to one integrated window or user interface. As in the embodiment of FIG. 4, the first distribution information 330 may be visualized in the second assistant information 300, and the second distribution information 430 may be visualized in third assistant information 400.

In an embodiment of the present invention, the visualization information may be displayed through one window or user interface in an integrated manner, or may be displayed through two or more windows or user interfaces. For example, the second assistant information 300 including the first distribution information 330 may be displayed via a first display, and the third assistant information 400 including the second distribution information 430 may be displayed via a second display.

In another embodiment of the present invention, the first distribution information 330 and the second distribution information 430 may be overlaid and visualized on a plane formed by one horizontal axis and one vertical axis. In this case, the first distribution information 330 and the second distribution information 430 may be visualized by different visualization elements so that they can be compared with each other.

The apparatus may generate first assistant information 340 or 440 based on a first threshold for signal intensity values within the first target region 110 and the second target region 210. The apparatus may generate the second assistant information 300 as a part of visualization information based on the first distribution information 330 and the first assistant information 340/the first threshold, and may also generate the third assistant information 400 as a part of visualization information based on the second distribution information 430 and the first assistant information 440/the first threshold.

The first distribution information 330 may be a histogram of signal intensity values corresponding to pixels/voxels within the first target region 110 represented on the horizontal axis 310 representative of signal intensity values and the vertical axis 320 corresponding to the number or distribution of the pixels/voxels of the signal intensity values. The second distribution information 430 may be a histogram of signal intensity values corresponding to pixels/voxels within the first target region 210 represented on the horizontal axis 410 representative of signal intensity values and the vertical axis 420 corresponding to the number or distribution of the pixels/voxels of the signal intensity values.

The signal intensity values to be applied to the first target region 110 and the second target region 210 included in the second assistant information 300 and the third assistant information 400 may be classified into the first interval 350 or 450 corresponding to the first state and the second interval 352 or 452 corresponding to the second state based on the first threshold.

The apparatus may generate the second assistant information 300, including the first assistant information 340 while including at least one of the first interval distribution information formed by the pixels/voxels within the first target region 110 to correspond to the first interval 350 of the signal intensity values and the second interval distribution information formed by the pixels/voxels within the first target region 110 to correspond to the second interval 352 of the signal intensity values, as a part of the visualization information. The first interval distribution information may be a segment corresponding to the first interval 350 of the first distribution information 330, and the second interval distribution information may be a segment corresponding to the second interval 352 of the first distribution information 330.

The apparatus may generate third assistant information 400, including the first assistant information 440 while including at least one of third interval distribution information formed by the pixels/voxels within the second target region 210 to correspond to the first interval 450 of the signal intensity values and fourth interval distribution information formed by the pixels/voxels within the second target region 210 to correspond to the second interval 452 of the signal intensity values, as part of visualization information. The third interval distribution information may be a segment corresponding to the first interval 450 of the second distribution information 430, and the fourth interval distribution information may be a segment corresponding to the second interval 452 of the second distribution information 430.

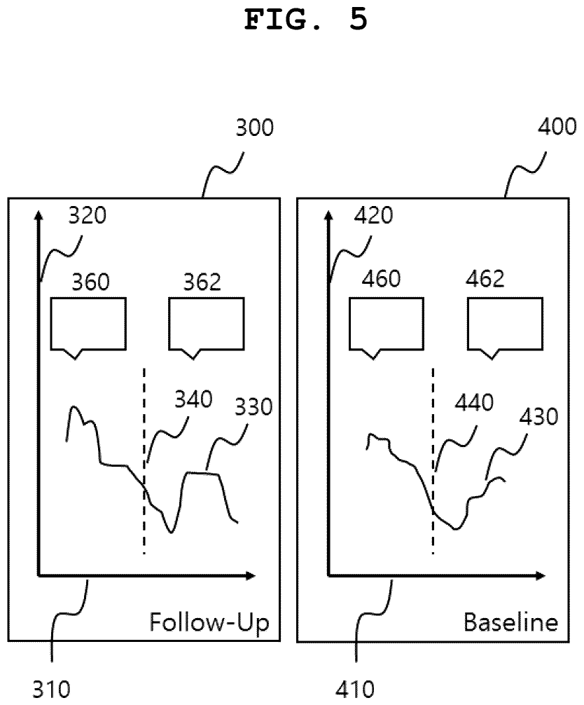

FIG. 5 is a diagram showing an example of assistant information visualized for medical image follow-up examination by a medical image diagnosis assistant apparatus according to an embodiment of the present invention.

The apparatus may generate at least one of first interval distribution quantification information 360 formed by the pixels/voxels within the first target region 110 to correspond to the first interval of the signal intensity values and second interval distribution quantification information 362 formed by the pixels/voxels within the first target region 110 to correspond to the second interval of the signal intensity values based on the first distribution information 330 and the first assistant information 340/the first threshold. The apparatus may generate the second assistant information 300, including the first assistant information 340 while including at least one of the first interval distribution quantification information 360 and the second interval distribution quantification information 362, as part of the visualization information.

The apparatus may generate at least one of third interval distribution quantification information 460 formed by the pixels/voxels within the second target region 210 to correspond to the first interval of the signal intensity values and fourth interval distribution quantification information 462 formed by the pixels/voxels within the second target region 210 to correspond to the second interval of the signal intensity values based on the second distribution information 430 and the first assistant information 440/the first threshold. The apparatus may generate the third assistant information 400, including the first assistant information 440 while including at least one of the third interval distribution quantification information 460 and the second interval distribution quantification information 462, as part of the visualization information.

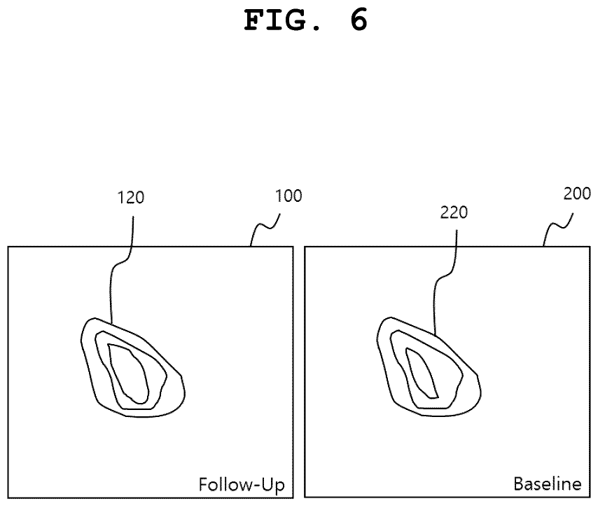

FIG. 6 is a diagram showing an example of a medical image visualized in association with assistant information for medical image follow-up examination by a medical image diagnosis assistant apparatus according to an embodiment of the present invention.

Referring to FIG. 6, there is shown an embodiment in which regions corresponding to individual histogram intervals (brightness value intervals) of the target region 120 or 220, which is a lesion/finding/organ region, are divided and marked on the medical image 100 or 200.

The apparatus may generate at least one of first overlay visualization information on the first medical image 100 for at least one first sub-region within the first target region 120 corresponding to the first interval 350, and second overlay visualization information on the first medical image 100 for at least one second sub-region within the first target region 120 corresponding to the second interval 352.

In the same manner, the apparatus may generate at least one of third overlay visualization information on the first medical image 200 for at least one first sub-region within the second target region 220 corresponding to the first interval 450, and fourth overlay visualization information on the first medical image 200 for at least one second sub-region within the second target region 220 corresponding to the second interval 452.

The first target region 120 may be segmented into sub-regions corresponding to a plurality of brightness value (signal intensity value) intervals, and the sub-regions may be divided and visualized, as shown in FIG. 6.

The second target region 220 may be segmented into sub-regions corresponding to a plurality of brightness value (signal intensity value) intervals, and the sub-regions may be divided and visualized, as shown in FIG. 6.

According to an embodiment of the present invention, the first target region 120 and second target region 220 of FIG. 6 segmented into sub-regions corresponding to brightness value intervals and visualized may be alternative embodiments of the second assistant information 300 and the third assistant information 400.

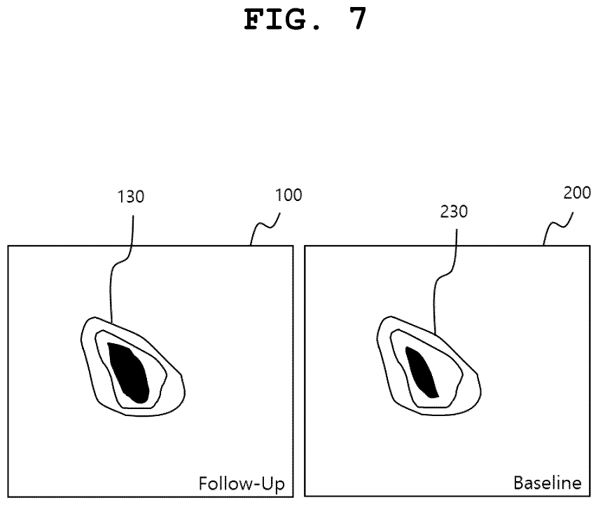

FIG. 7 is a diagram showing an example of a medical image visualized in association with assistant information for medical image follow-up examination by a medical image diagnosis assistant apparatus according to an embodiment of the present invention.

When a user input for the first interval 350 associated with the second assistant information 300 is recognized, the apparatus may generate first overlay visualization information for a sub-region corresponding to the first interval 350. In contrast, when a user input for the second interval 352 associated with the second assistant information 300 is recognized, the apparatus may generate second overlay visualization information for a sub-region corresponding to the second interval 352. For example, when a user input for the first interval 350 region of the second assistant information 300 or the first interval distribution quantification information 360 corresponding to the first interval 350 is recognized, it may be considered that there is a user input for the first interval 350, and the first overlay visualization information may be generated.

According to another embodiment of the present invention, the apparatus may generate the first overlay visualization information associated with the first interval 350 and the second overlay visualization information associated with the second interval 352 according to a predetermined routine without any user input.

The first target region 130 shown in FIG. 7 shows an embodiment that is visualized along with the first overlay visualization information that is visualized such that a sub-region corresponding to the first interval 350 can be emphasized (segmented from other sub-regions).

In the same manner, the second target region 230 shows an embodiment that is visualized along with the third overlay visualization information that is visualized such that a sub-region corresponding to the first interval 450 can be emphasized (segmented from other sub-regions).

When a user input corresponding to the second intervals 352 and 452 is recognized, the first target region 130 may be visualized along with the second overlay visualization information and the second target region 230 may be visualized along with the fourth overlay visualization information so that sub-regions corresponding to the second intervals 352 and 452 can be emphasized.

In this case, while specific sub-regions corresponding to specific brightness value intervals in the target region are emphasized and visualized on a medical image, the specific brightness value intervals corresponding to the specific sub-regions may also be emphasized and visualized in the assistant information 300 and 400. In other words, the interval information of the assistant information 300 and 400 may be emphasized and visualized in synchronization with overlay visualization information in which sub-regions on the medical image are emphasized.

According to an embodiment of the present invention, the first target region 130 and second target region 230 of FIG. 7 in which sub-regions corresponding to specific brightness value intervals are divided, emphasized, and visualized may be alternative embodiments of the second assistant information 300 and the third assistant information 400.

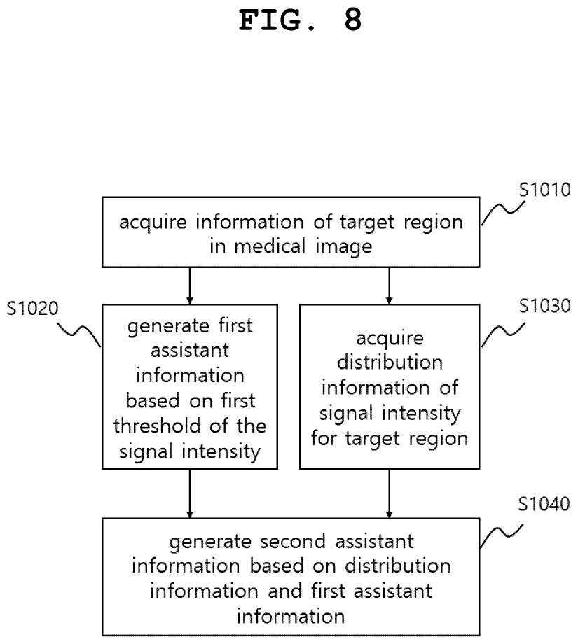

FIG. 8 is an operational flowchart showing a medical image diagnosis assistant method according to an embodiment of the present invention.

The medical image diagnosis assistant method according to an embodiment of the present invention may be performed by a computing system including a processor. The medical image diagnosis assistant method according to an embodiment of the present invention may include: step S1010 of acquiring information about at least one target region in a medical image; step S1030 of acquiring distribution information about the distribution of signal intensity values within the target region; step S1020 of generating first assistant information based on a first threshold for the signal intensity values within the target region; and step S1040 of generating second assistant information based on the distribution information and the first assistant information.

In this case, the signal intensity values within the target region may be classified into a first interval corresponding to a first state and a second interval corresponding to a second state based on a first threshold.

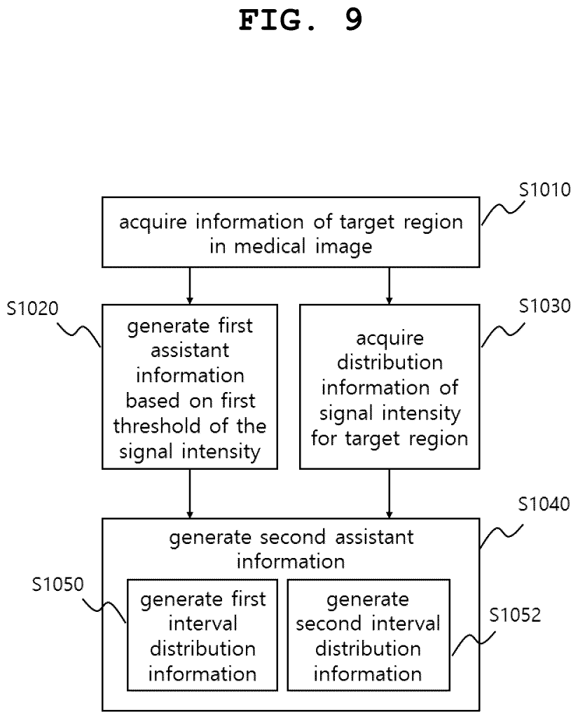

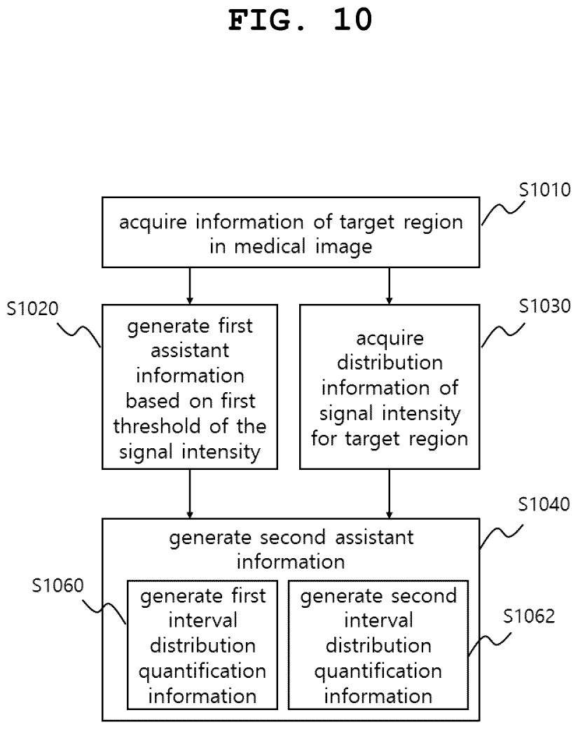

FIGS. 9 and 10 are operational flowcharts each showing in detail an example of a partial process of the medical image diagnosis assistant method of FIG. 8.

Referring to FIG. 9, step S1040 of generating second assistant information may include: step S1050 of generating first interval distribution information formed by the pixels/voxels within the target region corresponding to the first interval of the signal intensity values; and step S1052 of generating second interval distribution information formed by the pixels/voxels within the target region corresponding to the second interval of the signal intensity values.

In step S1040 of generating second assistant information of the medical image diagnosis assistant method according to an embodiment of the present invention, there may be generated second assistant information including at least one of the first interval distribution information and the second interval distribution information.

Referring to FIG. 10, step S1040 of generating second assistant information may include: step S1040 of generating first interval distribution quantification information, which is quantification information about a distribution corresponding to the first interval of the signal intensity values of the pixels/voxels within the target region; and step S1062 of generating second interval distribution quantification information, which is quantification information about a distribution corresponding to the second interval of the signal intensity values of the pixels/voxels within the target region.

In step S1040 of generating second assistant information of the medical image diagnosis assistant method according to an embodiment of the present invention, there may be generated second assistant information including at least one of the first interval distribution information and the second interval distribution information.

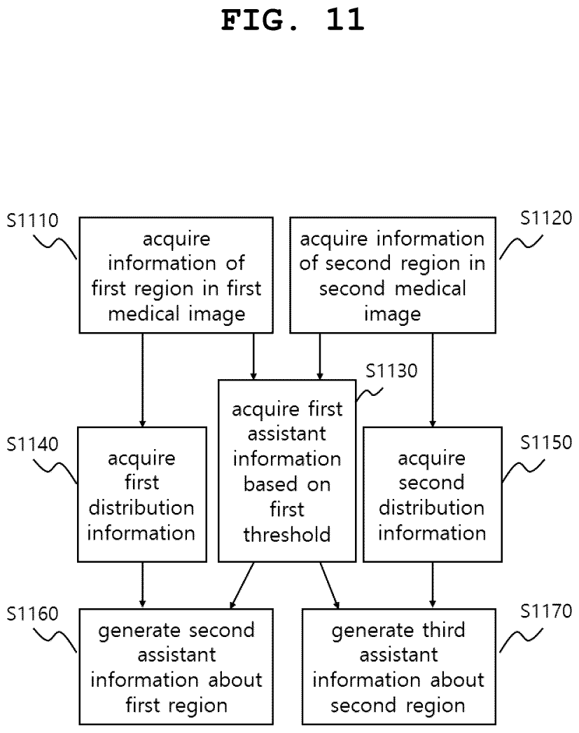

FIG. 11 is an operational flowchart showing a medical image diagnosis assistant method according to an embodiment of the present invention.

The medical image diagnosis assistant method according to an embodiment of the present invention may include: step S1110 of acquiring information about at least one first region in a first medical image; step S1140 of acquiring first distribution information about the distribution of signal intensity values within the first region; and step S1160 of generating second assistant information about the first region based on the first distribution information.

The medical image diagnosis assistant method according to an embodiment of the present invention may include: step S1120 of acquiring information about at least one second region in a second medical image; step S1150 of acquiring second distribution information about the distribution of signal intensity values within the second region; and step S1170 of generating third assistant information based on the second distribution information.

In the medical image diagnosis assistant method according to an embodiment of the present invention, there may be generated visualization information including the second assistant information and the third assistant information. The visualization information may be displayed through one window or user interface in an integrated manner, or may be displayed through two or more windows or user interfaces.

The medical image diagnosis assistant method according to an embodiment of the present invention may further include step S1130 of acquiring first assistant information applied to both the first region and the second region based on a first threshold for the signal intensity values. In this case, in step S1160, second assistant information for the first region may be generated based on the first distribution information and the first assistant information. In step S1170, third assistant information may be generated based on the second distribution information and the first assistant information.

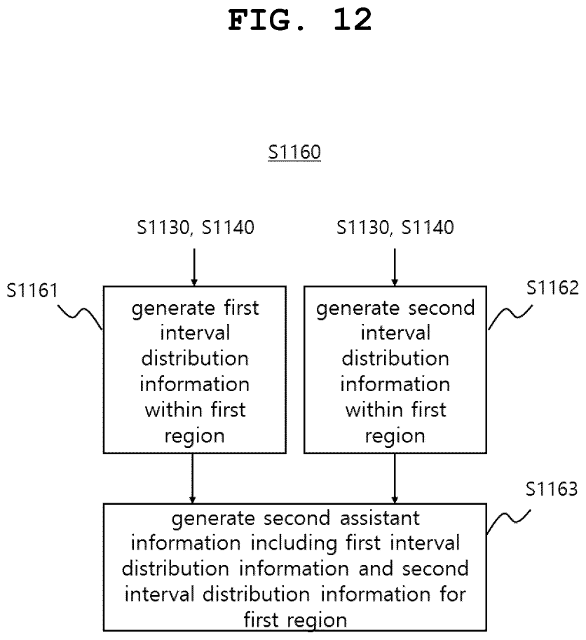

FIGS. 12 to 15 are operational flowcharts each showing in detail an example of a partial process of the medical image diagnosis assistant method of FIG. 11.

Referring to FIG. 12, step S1160 of generating second assistant information about the first region may include: step S1161 of generating first interval distribution information within the first region; step 1162 of generating second interval distribution information within the first region; and step S1163 of generating second assistant information including the first interval distribution information and the second interval distribution information for the first region.

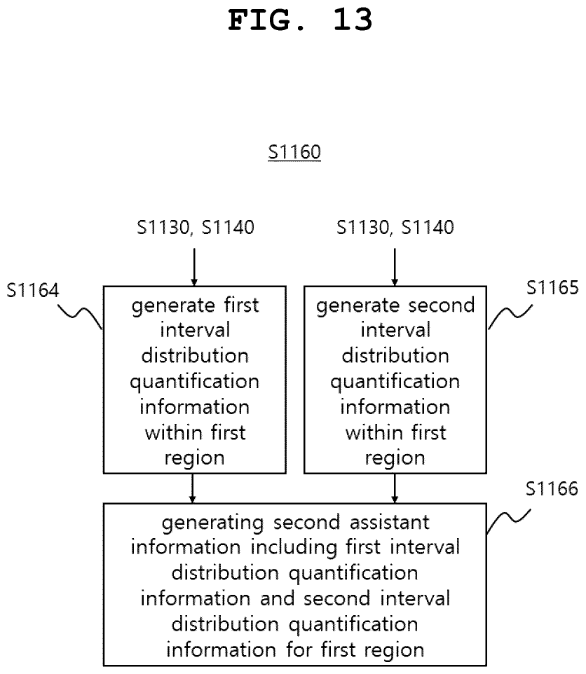

Referring to FIG. 13, step S1160 of generating second assistant information about the first region may include: step S1164 of generating first interval distribution quantification information within the first region; step 1165 of generating second interval distribution quantification information within the first region; and step S1166 of generating second assistant information including the first interval distribution quantification information and the second interval distribution quantification information for the first region.

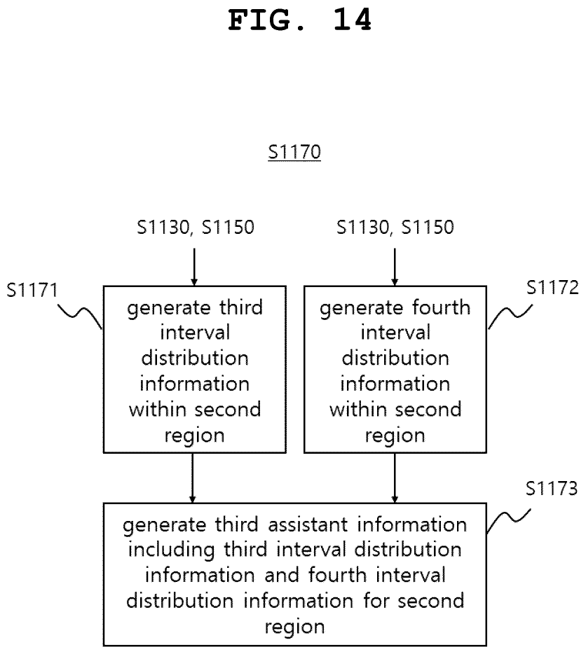

Referring to FIG. 14, step S1170 of generating third assistant information about the second region may include: step S1171 of generating third interval distribution information within the second region; step 1172 of generating fourth interval distribution information within the second region; and step S1173 of generating third assistant information including the third interval distribution information and the fourth interval distribution information for the second region.

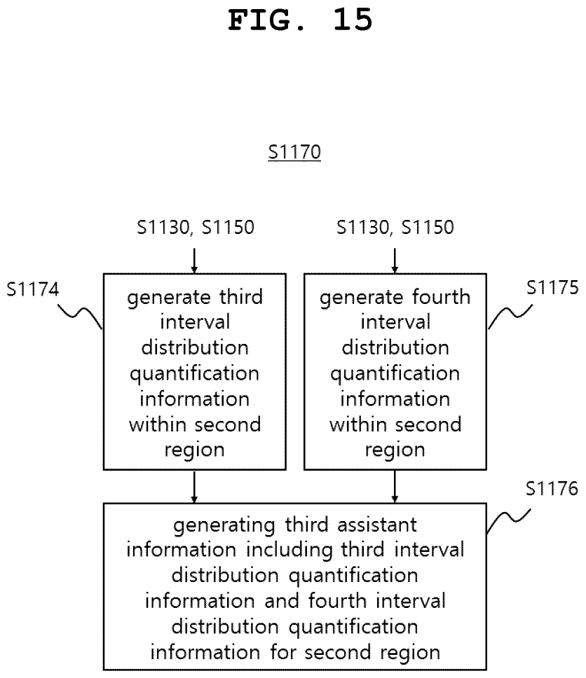

Referring to FIG. 15, step S1170 of generating third assistant information for the second region may include: step S1174 of generating third interval distribution quantification information within the second region; step S1175 of generating fourth interval distribution quantification information within the second region; and step S1176 of generating third assistant information including the third interval distribution quantification information and the fourth interval distribution quantification information for the second region.

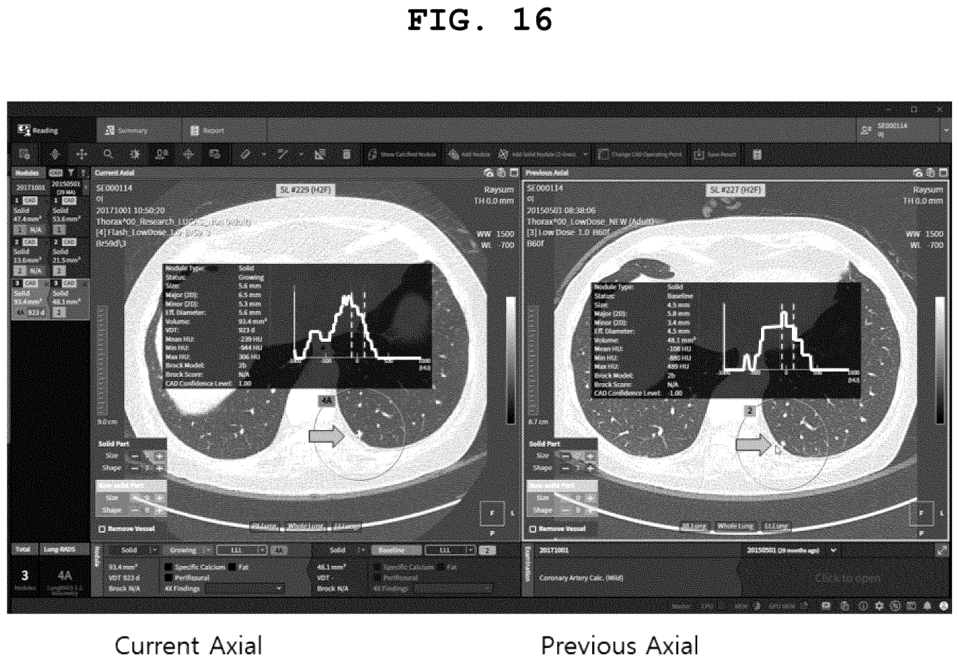

FIG. 16 is a drawing showing an example of medical image follow-up examination and assistant information according to an embodiment of the present invention.

Referring to FIG. 16, an axial image of a current image and an axial image of a previous image are displayed. A lung nodule detected in the current image and a lung nodule detected in the previous image are emphasized and displayed such that they can be compared with each other.

A histogram report in a translucent window format may be visualized on each of the axial image of the current image and the axial image of the previous image. The histogram report may be visualized for the CT brightness value interval of [−1000 HU, 500 HU] so that a user who is a medical professional can check the characteristics of the lung nodule. In particular, reference lines may also be visualized as first assistant information in the CT brightness value interval of [−100 HU, +100 HU] so that the user can identify whether the lung nodule is solid, part-solid, or non-solid.

In other words, in FIG. 16, when an ROI is a lung nodule, the distributions of brightness values of the ROI over time are visualized for comparison.

The configuration of the present invention described through the embodiments of FIGS. 1 to 16 is as follows.

In an embodiment of the present invention, the distribution of HU scale values of a CT image may be shown for pixels/voxels within a segmented region.

The segmented region may be referred to as an ROI.

The distribution of HU scale values may be a histogram.

The horizontal axis of the histogram may represent HU scale brightness value, and the vertical axis thereof may represent the number of pixels/voxels corresponding to each brightness value.

The ROI, which is the segmented region, may be a body part.

The ROI, which is the segmented region, may be an organ such as the heart, a lung, the liver, the stomach, or the cardiovascular system.

The ROI, which is the segmented region, may be a lesion such as a tumor, a lung nodule, a calcified region of the cardiovascular system, or a polyp.

A histogram of brightness values of the segmented region may be shown for each of the same ROIs corresponding to each other in each of a baseline image and a follow-up image.

In order to find the same ROIs corresponding to each other in the baseline image and the follow-up image, the baseline image and the follow-up image may be registered to each other.

Each of the pixels/voxels of the ROI of the baseline image and each of the pixels/voxels of the ROI of the follow-up image may be registered to each other.

The distribution of brightness values of the pixels/voxels of the ROI of the baseline image and the distribution of brightness values of the pixels/voxels of the ROI of the follow-up image are visualized such that they are compared with each other, so that the clinical characteristics of the ROI of the baseline image and the clinical characteristics of the ROI of the follow-up image can be compared with each other.

For example, when the ROI is an organ, the severity of a disease in the organ, the spread of the disease in the organ, the risk of the disease in the organ, the spread of a disease region in the organ, and/or the like may be clinically diagnosed by comparing the distributions of brightness values in the baseline image and the follow-up image.

For example, when the ROI is a lesion, the severity of the lesion, the spread of a severe region within the lesion, the risk within the lesion, and the spread of a risky region within the lesion may be clinically diagnosed by comparing the distributions of brightness values in the baseline image and the follow-up image.

In an embodiment of the present invention, clinical diagnosis is a user's role, and the interface of the present invention visualizes the distributions of brightness values in a baseline image and a follow-up image, thereby assisting a user in comparing the distributions and making clinical diagnose.

In an embodiment of the present invention, clinical diagnosis is a user's role, and the interface of the present invention visualizes the distributions of brightness values in a baseline image and a follow-up image and additionally provides quantitative measurement information about the distributions of brightness values within ROIs in the baseline image and the follow-up image, thereby providing diagnosis assistant information when a user makes clinical diagnose.

As an embodiment, when the ROI is a nodule in a lung region, it is made easy to be aware of a change in the brightness value of the nodule between a baseline image and a follow-up image, thereby providing information such as whether the nodule is a non-solid, part-solid, or solid nodule, and/or whether the nodule becomes malignant to a solid nodule. For example, when a case having a high brightness value corresponds to a solid region, it may be predicted that the possibility of becoming malignant is high when the number of pixels/voxels having a high brightness value increases.

When the distribution of brightness values is visualized, the range of brightness values to be visualized may be selected using a window specialized for the follow-up diagnosis of an organ or a lesion. For example, in the case of a lung nodule, the distribution of brightness values may be visualized in the window range of [−1000 HU, +500 HU]. In addition, −100 HU, and +100 HU reference lines are marked for critical values that are selected from the visualized brightness values and can give clinical significance to the follow-up diagnosis of an organ or a lesion, thereby being helpful in clinically determining the type, severity, and risk of the organ or the lesion.

A configuration for visualizing the distribution of signal intensity/brightness values in a CT/MR image according to an embodiment of the present invention may be combined with the following configurations.
1) The configuration for visualizing may be combined with a follow-up examination function, or
2) The configuration for visualization may be combined with a menu for checking the validity of segmentation results and may be provided through the corresponding menu.

In the case of a follow-up examination function, it is different from conventional technologies in that it shows a baseline region and a histogram of a follow-up region together.

In addition to histogram analysis/visualization, inter-lesion registration and measurement configurations required for a follow-up examination function may be combined.
3) The segmented ROI may be a body part or organ of the human body. Alternatively, the ROI may be a lesion, particularly a lung nodule or a lung tumor.

In this case, a process in which a user identifies the type of nodule may be assisted by marking −100 HU and 100 HU reference lines. The range of HU values marked on the horizontal axis of a histogram is an embodiment specialized for a corresponding lesion, and may be optimized within the interval of [−1000 HU, 500 HU] in the case of a lung nodule as an example.

In an embodiment in which the distribution of brightness values is visualized through a histogram, one or more reference lines for brightness values may be provided, thereby assisting a user in identifying the type or state of an ROI. In this case, the results of quantitative analysis of a plurality of brightness value intervals divided by one or more reference lines may be visualized together with the reference lines.

Alternatively, a medical image analysis module may determine the type or state of an ROI based on reference lines, and may visualize the results of the determination together with the reference lines. In this case, the results of quantitative analysis of a plurality of brightness value intervals divided by one or more reference lines may be visualized together with the reference lines and the results of determination of the type or state of an ROI. In this case, the results of the determination may further include prediction/inference information about a change in the type or state of an ROI.

For example, when the ROI is a lung nodule, there may be provided one or more brightness value reference lines that facilitate the identification of the types of nodules (solid, non-solid, and part-solid types).

When the distribution of brightness values is visualized in the form of a contour map of brightness values instead of a histogram, a plurality of regions divided based on the reference lines for brightness values may be visualized using visual elements that are distinguished from each other. In this case, at least one of the regions divided by the reference lines, the results of quantitative analysis of each of the regions, and the results of determination of the type or state of an ROI may also be visualized.
4) In the case of a specific organ or lesion, a CT value window for a histogram and/or contour map may be determined to meet a clinical diagnosis purpose.
5) In an embodiment of a CT or MRI histogram combined with a follow-up examination function, it may be applicable even when the ROI is not a lesion, nodule, or tumor but a general organ.

The prior art analyzes a histogram and thus provides a kurtosis map or skewness map. There is not provided a means by which a user can verify these calculation results. In an embodiment of the present invention, there may be provided a user interface (UI) configured to assist a medical staff in reviewing the progress of a disease in an organ by displaying the distribution of all pixels within a region.

There may be added a function of attracting the attention of a medical staff or assisting determination by displaying an interval/part in which a change in brightness value appears in follow-up examination.
6) The histogram visualization function may also be invoked through a menu that is provided to check the validity of segmentation results.

In connection with 6) above, there may be provided a menu that can check the validity of the results of segmentation of both a baseline region and a follow-up region.

The detection of a nodule or a tumor may be achieved by performing segmentation based on the HU values of CT.

A detection process may be implemented via segmentation or thresholding.

The distribution of CT brightness values of pixels within the ROI may be provided through a menu that allows a user (a medical staff or medical imaging professional) to review the validity of an ROI (which may be an organ or a lesion) derived as a result of segmentation or thresholding and is provided for the ROI.

The distribution of brightness values may be a histogram, or may be visualized through a contour map.

For example, when the ROI is a lesion or a tumor, there may be provided a UI that assists a user in easily determining whether a nodule or the tumor is malignant by allowing the user to easily become aware of a change in the brightness value in follow-up examination.

For example, when the ROI is a lung nodule, a change in the brightness value may be clinically interpreted as being solidified from a non-solid or part-solid state to a solid state.

For example, when the HU value of a solid region is higher than that of a non-solid region within a nodule, the nodule may be interpreted as being solidified from a non-solid or part-solid state to a solid state in the case where there are more pixels having a higher HU value in the follow-up nodule than in the baseline nodule.

In lung cancer screening (LCS), changes in the size of a nodule before the occurrence of lung cancer are generally tracked. In an embodiment of the present invention, when the size of a nodule does not change but the distribution of HU values of pixels changes, notification that the nodule is solidified and thus the malignancy of the nodule is in progress may be provided to a user.

In an embodiment in which the ROI is an organ, there may be provided a UI that assists a user to easily determine the progress of a disease in the organ by allowing the user to easily be aware of a change in the brightness value in follow-up examination.

When the ROI is an organ, there may be provided a UI that provides a user with the hardening of the liver, an increase in a lung LAA region, an increase in a cardiovascular calcification region, or the like through a histogram so that the user can easily be aware of it.

When the ROI is an organ, the distributions of brightness values may be compared or changes in the distribution may be visualized, so that changes in the size of the organ can be visualized such that a user can easily be aware of them and so that the severity of a disease can be quantified. When the size of the organ does not change, the distributions of brightness values inside the organ may be compared or changes in the distribution may be visualized, so that changes in the components inside the organ or the severity of a disease can be visualized such that a user can easily be aware of them and so that the severity of the disease can be quantified.

Option 1: Currently, a histogram is displayed based on the HU brightness values of pixels within a region that is detected as a nodule. When a doctor corrects the nodule region (through an editing menu), a corrected histogram may be displayed for the corrected region.

Currently, there is employed an embodiment of a histogram in which the horizontal axis represents HU values and the vertical axis represents the numbers of pixels. However, there may be employed a UI that displays a corresponding nodule region like contour lines while varying the color thereof according to the HU value.

For size comparison, a contour map may be overlaid after a previous/past lesion has been displayed on the same scale. A UI such as a contour map may provide insight into a change in the shape or size together with a change in the intensity.

There may be added a function of attracting the attention of a medical staff or assisting determination by displaying an interval/part in which a change in brightness value appears in follow-up examination.

There may be added a function of attracting the attention of a medical staff or assisting determination by displaying an interval/part in which a change in the frequencies of brightness values in follow-up examination is significant.

There may be displayed an interval/part in which a change in the brightness value between a baseline ROI and a follow-up ROI is equal to or larger than a threshold.

There may be displayed an interval/part in which a change in the frequencies of brightness values between a baseline ROI and a follow-up ROI is equal to or larger than a threshold.

After performing image processing and image analysis on medical images, the processor of a medical image diagnosis assistant apparatus according to an embodiment of the present invention may generate the results of each step of the image processing and the image analysis as at least one of an interim result and a final result, may generate the final result as a temporary result image according to standardized specifications such as DICOM and HL7, may generate detailed analysis information including the interim result and the final result, may generate a link image such as a QR image for the detailed analysis information, and may generate an image connected to at least one of the interim result and the final result by adding the link image to the temporary result image.

The processor of the medical image diagnosis assistant apparatus according to an embodiment of the present invention may allow image information and non-image information for the generated interim result and final result to be included in the detailed analysis information.

The processor of a medical image diagnosis assistant apparatus according to an embodiment of the present invention may recognize a link image (for example, a QR code image) displayed on a linked image, may request detailed analysis information from a medical image analysis server through the link image, and may visualize the detailed analysis information received from the server on a display. In this case, the detailed analysis information may be generated to include the interim result and final result of a process in which the results of image processing and image analysis performed on medical images are generated as the linked image.

The processor of a medical image diagnosis assistant apparatus according to an embodiment of the present invention may visualize detailed analysis information together with a menu to which a user's feedback of approval or rejection of at least one of an interim result and a final result can be input.

When the user approves the at least one result, the processor of a medical image diagnosis assistant apparatus according to an embodiment of the present invention may store the user's approval in association with an original medical image, a medical image linked by a link image, and detailed analysis result in an internal or external database.

The processor of a medical image diagnosis assistant apparatus according to an embodiment of the present invention may link a work environment menu, via which a user can manually modify at least one of an interim result and a final result instead of a user's approval, with a link image, and may provide the work environment menu together with detailed analysis information.

The detailed analysis information may include the results of preprocessing of the original medical image as an interim result, and the processor of a medical image diagnosis assistant apparatus according to an embodiment of the present invention may include the results of quantitative analysis of the original medical image, generated based on the results of preprocessing, as a final result. The results of preprocessing may be the results of segmentation of a specific organ or lesion. The results of preprocessing may be visualized through a separate screen on which they can be compared with the results of quantitative analysis, and the results of preprocessing and the results of quantitative analysis may be overlaid and displayed on a single screen.

The detailed analysis information may include the results of object identification of the original medical image as an interim result, and the processor of a medical image diagnosis assistant apparatus according to an embodiment of the present invention may include the results of filtering, obtained by applying a threshold to the results of object identification, as a final result.

The processor of a medical image diagnosis assistant apparatus according to an embodiment of the present invention may link a menu, via which a medical image can be edited or a new medical image, in which the settings of the medical image have been adjusted, can be generated, with a link image, and may provide the menu together with detailed analysis information.

When the medical image is at least one of reconstructed and reformatted images of the original medical image, the processor of a medical image diagnosis assistant apparatus according to an embodiment of the present invention may link a work environment menu, via which at least one of new reconstructed and reformatted images can be generated as a new medical image by adjusting at least one of the range, angle, viewpoint, and option in which at least one of the reconstructed and reformatted images is generated, with a link image, and may provide the work environment menu together with detailed analysis information.

When at least one medical image includes a report representing the results of image analysis, the processor of a medical image diagnosis assistant apparatus according to an embodiment of the present invention may link a work environment menu, via which a new report can be generated by adjusting at least one parameter used to generate a report, with a link image, and may provide the work environment menu together with detailed analysis information.

When the detailed analysis information includes the results of object identification for the original medical image, the processor of a medical image diagnosis assistant apparatus according to an embodiment of the present invention may link a work environment menu, via which a threshold applied to the results of object identification can be modified, the results of filtering obtained by applying the modified threshold can be generated as new detailed analysis information, and the results of object identification before the application of the threshold can be manually verified, with a link image, and may provide the work environment menu together with detailed analysis information.

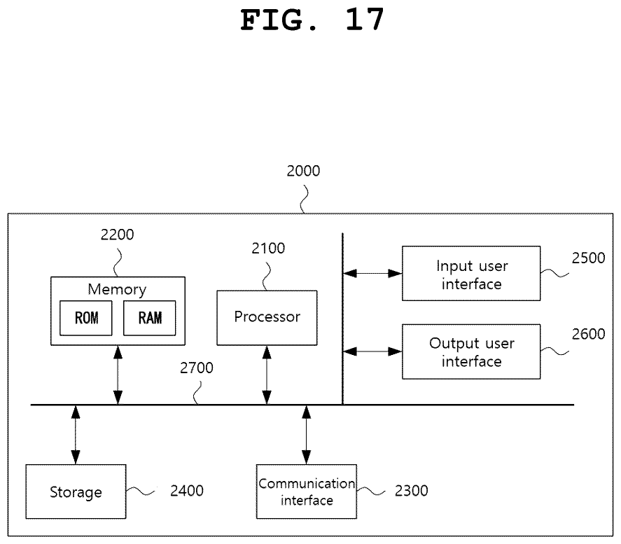

FIG. 17 is a conceptual block diagram showing a generalized medical image diagnosis assistant apparatus or computing system capable of performing at least a part of the processes of FIGS. 1 to 16 according to an embodiment of the present invention.

Although omitted in the drawings in connection with the embodiments of FIGS. 1 to 16, a processor and memory are electronically connected to the individual components, and the operations of the individual components may be controlled or managed by the processor.

At least some processes of the medical image diagnosis assistant method according to an embodiment of the present invention may be executed by the computing system 2000 of FIG. 17.

As shown in FIG. 17, the computing system 2000 according to an exemplary embodiment of the present disclosure may be configured to include a processor 2100, a memory 2200, a communication interface 2300, a storage device 2400, an input interface 2500, an output interface 2600, and a bus 2700.

The computing system 2000 according to an exemplary embodiment of the present disclosure may include the at least one processor 2100 and the memory 2200 storing instructions instructing the at least one processor 2100 to perform at least one step. At least some steps of the method according to exemplary embodiments of the present disclosure may be performed by the at least one processor 2100 loading the instructions from the memory 2200 and executing them.

The processor 2100 may mean a central processing unit (CPU), a graphics processing unit (GPU), or a dedicated processor on which the methods according to exemplary embodiments of the present disclosure are performed.

Each of the memory 2200 and the storage device 2400 may include at least one of a volatile storage medium and a non-volatile storage medium. For example, the memory 2200 may include at least one of a read only memory (ROM) and a random access memory (RAM).

In addition, the computing system 2000 may include the communication interface 1300 that performs communication through a wireless network.

In addition, the respective components included in the computing system 2000 may be connected by the bus 2700 to communicate with each other.

For example, the computing system 2000 including the processor 2100 of the present disclosure may be a desktop computer, a laptop computer, a notebook, a smart phone, a tablet PC, a mobile phone, a smart watch, a smart glass, e-book reader, a portable multimedia player (PMP), a portable gaming device, a navigation device, a digital camera, a digital multimedia broadcasting (DMB) player, a digital audio recorder, a digital audio player, a digital video recorder, a digital video player, a personal digital assistant (PDA), and the like having communication capability.

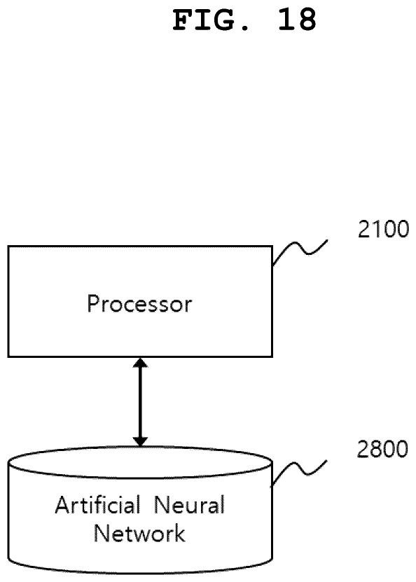

FIG. 18 is a conceptual diagram including a processor and an artificial neural network as the internal structure of a generalized medical image diagnosis assistant apparatus or computing system according to an embodiment of the present invention.

Some of the components that are included in the apparatus or computing system of FIG. 18 and shown in FIG. 17 are omitted for the brevity of description. The processor 2100 of FIG. 18 may be connected to the artificial neural network 2800 via a bus 2700. A weight matrix constituting the artificial neural network 2800 may be stored in memory 2200 and/or a storage device 2400, and activation parameters generated during an artificial neural network operation may be stored in the memory 2200 and/or the storage device 2400.

The weights and activation parameters constituting the artificial neural network 2800 may be stored in a separate device (not shown) other than the memory 2200 and/or the storage device 2400, and the separate device may be connected to the processor 2100 and perform an artificial neural network operation under the control of the processor 2100. The artificial neural network operation may include data input/output between the processor 2100 and the artificial neural network 2800 and logical/arithmetic operations that are performed during a training process, an inference process, and/or a result output/generation process in order to perform the method according to the embodiment of the present invention. The training process, inference process, and/or result output/generation process of the artificial neural network 2800 may be executed under the control of the processor 2100.

According to an embodiment of the present invention, the means, by which a medical professional can verify the results of detection of a specific organ, lesion, or finding, can be provided as assistant information.

According to an embodiment of the present invention, the means, by which a medical professional can obtain additional information about the presence/absence, progress, and severity of a disease for a specific organ, lesion, or finding, may be provided as assistant information.

According to an embodiment of the present invention, in a state in which a specific organ is segmented or a lesion or finding is detected, and is then visualized, information about whether a disease is actually present in the segmented organ or the detected lesion or finding, whether the disease is in progress, or whether the disease is severe can be provided together with assistant information that a medical professional, which is a user, can be aware of.

According to an embodiment of the present invention, there is provided the UI that facilitates the follow-up examination of the transition of a specific lesion or organ over time.

According to an embodiment of the present invention, there is provided the UI including visualization information that is effective for representing the type or state of a region of interest (ROI).

According to an embodiment of the present invention, there is provided the UI including a visualization means that is effective for representing a change in the type or state of an ROI.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. A medical image diagnosis assistant apparatus comprising a processor, wherein the processor is configured to:
acquire information about at least one target region in a medical image;
acquire distribution information about a distribution of signal intensity values within the target region;
generate first assistant information based on a first threshold for the signal intensity values within the target region; and
generate second assistant information based on the distribution information and the first assistant information.

2. The medical image diagnosis assistant apparatus of claim 1, wherein the signal intensity values within the target region are classified into a first interval corresponding to a first state and a second interval corresponding to a second state based on the first threshold.

3. The medical image diagnosis assistant apparatus of claim 2, wherein the second assistant information comprises at least one of first interval distribution information corresponding to the first interval of the signal intensity values within the target region or second interval distribution information corresponding to the second interval of the signal intensity values within the target region.

4. The medical image diagnosis assistant apparatus of claim 3, wherein the second assistant information further comprises at least one of a first visualization element representative of the first interval distribution information or a second visualization element representative of the second interval distribution information.

5. The medical image diagnosis assistant apparatus of claim 2, wherein the second assistant information comprises at least one of first interval distribution quantification information corresponding to the first interval of the signal intensity values within the target region or second interval distribution quantification information corresponding to the second interval of the signal intensity values within the target region.

6. The medical image diagnosis assistant apparatus of claim 5, wherein the first interval distribution quantification information comprises at least one of a percentile, maximum value, minimum value, mean value, mode value, or median value of pixels/voxels of signal intensity values within the target region corresponding to the first interval, and
wherein the second interval distribution quantification information comprises at least one of a percentile, maximum value, minimum value, mean value, mode value, or median value of pixels/voxels of signal intensity values within the target region corresponding to the second interval.

7. The medical image diagnosis assistant apparatus of claim 2, wherein the processor is further configured to generate at least one of:
first overlay visualization information on the medical image for at least one first sub-region within the target region corresponding to the first interval; or
second overlay visualization information on the medical image for at least one second sub-region within the target region corresponding to the second interval.

8. The medical image diagnosis assistant apparatus of claim 7, wherein the processor is further configured to:
generate the first overlay visualization information when a user input for the first interval associated with the second assistant information is recognized; and
generate the second overlay visualization information when a user input for the second interval associated with the second assistant information is recognized.

9. The medical image diagnosis assistant apparatus of claim 1, wherein the first threshold is associated with at least one of a presence/absence, progress, or severity of a disease associated with the target region.

10. The medical image diagnosis assistant apparatus of claim 1, wherein information about the target region is segmentation information about a boundary of the target region.

11. The medical image diagnosis assistant apparatus of claim 1, wherein the target region is a finding region detected in association with a disease or lesion in the medical image.

12. The medical image diagnosis assistant apparatus of claim 1, wherein the target region is a region obtained as a result of segmentation of an anatomical structure in the medical image.

13. The medical image diagnosis assistant apparatus of claim 1, wherein the processor is further configured to generate the first assistant information further including a second threshold value for the signal intensity values within the target region, and
wherein the signal intensity values within the target region are classified into a first interval corresponding to a first state, a second interval corresponding to a second state, and a third interval corresponding to a third state based on the first threshold and the second threshold.

14. The medical image diagnosis assistant apparatus of claim 1, wherein the distribution information is histogram information corresponding to a distribution of signal intensity values of pixels/voxels within the target region.

15. A medical image diagnosis assistant apparatus comprising a processor, wherein the processor is configured to:
acquire information about at least one first target region in a first medical image acquired at a first time for a subject;
acquire information about at least one second target region in a second medical image acquired at a second time for the subject;
acquire first distribution information about a distribution of signal intensity values within the first target region;
acquire second distribution information about a distribution of signal intensity values within the second target region; and
generate visualization information based on the first distribution information and the second distribution information.

16. The medical image diagnosis assistant apparatus of claim 15, wherein the processor is further configured to generate first assistant information based on a first threshold for signal intensity values in the first area and the second area,
wherein the signal intensity values within the first region and the second region are classified into a first interval corresponding to a first state and a second interval corresponding to a second state based on the first threshold,
wherein the processor is further configured to acquire at least one of first interval distribution information corresponding to the first interval for the signal intensity values within the first region or second interval distribution information corresponding to the second interval for the signal intensity values within the first region based on the first distribution information,
wherein the processor is further configured to generate, as a part of the visualization information, second assistant information including the first assistant information while including at least one of the first interval distribution information or the second interval distribution information,
wherein the processor is further configured to acquire at least one of third interval distribution information corresponding to the first interval for the signal intensity values within the second region or fourth interval distribution information corresponding to the second interval for the signal intensity values within the second region based on the second distribution information, and
wherein the processor is further configured to generate, as a part of the visualization information, third assistant information including the first assistant information while including at least one of the third interval distribution information or the fourth interval distribution information.

17. The medical image diagnosis assistant apparatus of claim 15, wherein the processor is further configured to generate first assistant information based on a first threshold for signal intensity values in the first area and the second area,
wherein the signal intensity values within the first region and the second region are classified into a first interval corresponding to a first state and a second interval corresponding to a second state based on the first threshold,
wherein the processor is further configured to acquire at least one of first interval distribution quantification information corresponding to the first interval for the signal intensity values within the first region or second interval distribution quantification information corresponding to the second interval for the signal intensity values within the first region based on the first distribution information,
wherein the processor is further configured to generate, as a part of the visualization information, second assistant information including the first assistant information while including at least one of the first interval distribution quantification information or the second interval distribution quantification information,
wherein the processor is further configured to acquire at least one of third interval distribution quantification information corresponding to the first interval for the signal intensity values within the second region or fourth interval distribution quantification information corresponding to the second interval for the signal intensity values within the second region based on the second distribution information, and
wherein the processor is further configured to generate, as a part of the visualization information, third assistant information including the first assistant information while including at least one of the third interval distribution quantification information or the fourth interval distribution quantification information.

18. A medical image diagnosis assistant method, the medical image diagnosis assistant method being performed by a computing system including a processor, the medical image diagnosis assistant method comprising:
acquiring information about at least one target region in a medical image;
acquiring distribution information about a distribution of signal intensity values within the target region;
generating first assistant information based on a first threshold for the signal intensity values within the target region; and
generating second assistant information based on the distribution information and the first assistant information.

19. The medical image diagnosis assistant method of claim 18, wherein the signal intensity values within the target region are classified into a first interval corresponding to a first state and a second interval corresponding to a second state based on the first threshold, and
wherein the generating second assistant comprises generating the second assistant information including at least one of first interval distribution information corresponding to the first interval of the signal intensity values within the target region or second interval distribution information corresponding to the second interval of the signal intensity values within the target region.

20. The medical image diagnosis assistant method of claim 18, wherein the signal intensity values within the target region are classified into a first interval corresponding to a first state and a second interval corresponding to a second state based on the first threshold, and
wherein the generating second assistant comprises generating the second assistant information including at least one of first interval distribution quantification information corresponding to the first interval of the signal intensity values within the target region or second interval distribution quantification information corresponding to the second interval of the signal intensity values within the target region.
